(19) United States
(12) Patent Application Publication
(10) Pub. No.: US 2008/0312356 A1
(43) Pub. Date: Dec. 18, 2008

(54) Vapor-deposited biocompatible coatings which adhere to various plastics and metal

(75) Inventors: Boris Kobrin, Dublin, CA (US); Michael T. Grimes, San Jose, CA (US); Jeffrey D. Chinn, Foster City, CA (US)

Correspondence Address: Peter Martine, Esq.;Martine Penilla & Gencarella, LLP, 710 Lakeway Drive, Suite 200, Sunnyvale, CA, 94085, US

(73) Assignee: Applied Mcrostructures, Inc.

(21) Appl. No.: 12/157,427

(22) Filed: Jun. 9, 2008

Related U.S. Application Data
(60) Provisional application No. 60/934,576, filed on Jun. 13, 2007.

(51) Int. Cl. A61L 27/34; C23C 16/02

(52) U.S. Cl. 523/113; 427/255.6

(57) ABSTRACT

A method of providing a biocompatible PEG-comprising coating on a substrate, without the use of an underlying adhesion layer. The coating is vapor deposited onto the substrate from a precursor which includes a PEG-derived moiety and an amino silane-containing functional group which reacts with the substrate. The substrate may be metal or plastic, where plastic excludes polyimide and polycarbonate. The substrate may be plasma treated prior to deposition of the PEG-comprising coating.

This application claims priority under U.S. Provisional Application Ser. No. 60/934,576 Jun. 13, 2007, and entitled “Poly(ethylene glycol) Vapor Deposited Coatings and Coated Articles”, which is hereby incorporated by reference in its entirety. In addition, this application is related to the following applications, under which priority is not claimed, but each of which is also hereby incorporated by reference in its entirety. U.S. application Ser. No. 11/445,706, filed Jun. 2, 2006 and titled “Apparatus and Method for Controlled Application of Reactive Vapors to produce Thin Films and Coatings” (Pub No. US 2006-0213441 A1), currently pending, which is a continuation application of U.S. application Ser. No. 10/759,857, filed Jan. 17, 2004 and titled: “Apparatus and Method for Controlled Application of Reactive Vapors to Produce Thin Films and Coatings”, now abandoned; U.S. application Ser. No. 10/912,656, filed Aug. 4, 2004 (Pub. No. US 2006-0029732 A1), and titled: “Vapor Deposited Functional Organic Coatings”, currently pending; U.S. application Ser. No. 11/112,664, filed Apr. 21, 2005 (Pub. No. US 2005-0271900 A1), and titled: “Controlled Deposition of Multilayered Coatings Adhered by an Oxide Layer”, currently pending; U.S. application Ser. No. 11/123,487, filed May 5, 2005 (Pub. No. US 2006-0251795 A1), and titled: “Controlled Vapor Deposition of Biocompatible Coatings for Medical Devices” currently pending; and, U.S. application Ser. No. 11/295,129, filed Dec. 5, 2005 (Pub No. US 2006-0088666 A1), and titled: “Controlled Vapor Deposition of Biocompatible Coatings Over Surface Treated Substrates”, currently pending.

### BACKGROUND OF THE INVENTION

1. Field

Vapor deposited coatings which comprise a derivative of PEG (polyethylene glycol), a method of applying the coatings, and articles with the coating applied.

2. Brief Description of the Background Art

This section describes background subject matter related to the disclosed embodiments of the present invention. There is no intention, either express or implied, that the background art discussed in this section legally constitutes prior art.

In the biological field, the surface characteristics of a substrate control the functioning of that substrate relative to fluids and other surfaces with which the substrate surface comes in contact. Since known living organisms rely heavily on the presence of water, the hydrophilicity or hydrophobicity of a given surface plays a major role in determining whether a medical device can perform well in the environment in which it is to function. The surface of the medical device must be designed to provide biocompatibility with fluids it is to contact in the environment, and may be designed to achieve a particular interaction with the fluids it contacts. The ability of a medical device to function either in-vivo or in-vitro depends on the surface presented by the medical device. For example, with respect to an implant which is used in medical applications, the ability of the implant to integrate into the location at which it is placed and to function in combination with surrounding tissues and fluids depends significantly on the hydrophilicity or hydrophobicity of the implant surface, and frequently depends on the presence or absence on the surface of the implant of chemical compounds having particular properties. With respect to a medical device surface used for chemical analysis, for example, the device must provide a functional surface which enables the particular analytical function.

It is widely recognized that the modification of biomaterial surfaces is a useful strategy for controlling protein adsorption and cell interactions with materials. Coating of materials, micro structures, and components with biocompatible films which can be used for bio and medical applications is frequently motivated by the desire to obtain a non-degrading bio-compatible surface with a specific anti-fouling surface property. To address the bio-fouling problem, much attention has been directed towards the development of chemical strategies for modifying materials' surfaces to improve their resistance to biological contamination. Poly(ethylene glycol) (PEG) and its derivatives which include the structural formula —(CH₂—CH₂—O)— are known to be bio-compatible. Derivatives which include an —OH functional end group are known to be useful in anti-fouling applications in pharmaceutical, medical diagnostics, protein chemistry, and related fields. PEG functionalized surfaces have been shown to exhibit reduced adsorption of macromolecules such as proteins, peptides, and some bacteria. PEGs are relatively inexpensive and can be obtained in molecular weights ranging from less than 200 to several thousands. Lower molecular weight PEGs are liquid while higher molecular weight PEGs are solid and are used in the form of powders or solutions (e.g. in laxatives, ophthalmic solutions, and other medical applications). Recent advances in Bio-MEMS, assays, and automated diagnostics techniques require a high degree of precision both in terms of composition and three dimensional structure of a coating which is to provide particular surface properties.

The liquid and vapor phase coating techniques used to provide functional PEG derivative surfaces frequently make use of functionalized silane-based groups for attachment to substrate surfaces. A chloromethoxy or chloroethoxy silane can attach a functionalized PEG derivative to a surface. The most commonly used surface reaction mechanism for a silane-based PEG precursor attachment from a vapor phase is reaction of the chlorosilane via hydrolysis with a substrate exhibiting surface hydroxyl groups. Reaction with hydroxyl groups provides covalent bonding, which is responsible for the relatively high mechanical and chemical stability of the vapor deposited PEG derivative films.

Miqin Zhang et al., in an article entitled “Hemocompatible Polyethylene Glycol Films on silicon”, published in Biomedical Microdevices, 1(1), pp. 81-87 (1998), describe the functionalization of polyethylene glycol (PEG) by SiCl₃ groups on its chain ends, and the reaction of the PEG organosilicon derivatives with hydroxylated groups on silicon surfaces. The reactant preparations and the attachment of a PEG derivative film onto silicon surfaces were carried out in a glass apparatus which prevented exposure to the atmosphere. Nitrogen was used as the isolation gas, and the precursor formation reactions were carried out in solutions, with attachment of the precursor to the silicon surface by contact of a precursor solution with the silicon surface.

In another article entitled “Proteins and cells on PEG immobilized silicon surfaces”, published in Biomaterials 19 (1998) pp. 953-960, Zhang et al. describe the modification of silicon surfaces by covalent attachment of self-assembled polyethylene glycol (PEG) film. Adsorption of albumin, fibrinogen, and IgG to PEG immobilized silicon surfaces was studied to evaluate the non-fouling and non-immunogenic properties of the surfaces. The adhesion and proliferation of human fibroblast and Hela cells onto the modified surfaces were investigated to examine their tissue biocompatibility. Coated PEG chains were said to show the effective depression of both plasma protein adsorption and cell attachment to the modified surfaces. The mechanisms accounting for the reduction of protein adsorption and cell adhesion on modified surfaces were discussed. (Abstract) This article is hereby incorporated by reference in its entirety. PEG was immobilized on silicon by the functionalization of a PEG precursor in the manner described in the article discussed above.

Despite the recognition that a PEG derivative coating precursor material, which presents a silane-comprising functional group for attachment purposes, is an excellent precursor material for depositing biocompatible coatings, the use of such precursor materials has been largely limited to substrates exhibiting a high concentration of hydroxyl groups, such as silicon, quartz, and various oxides. Chloromethoxy or chloroethoxy silane do not react well with acrylic, polyethylene, polypropylene, and other plastics which are frequently used in bio and medical applications, due to the relatively low concentration of hydroxyl groups on the surface of such materials. Specialized oxide adhesion layers used in combination with PEG derivative coating precursor materials have been proposed in the past to address this issue. These adhesion layers may include silicon and metal oxides, which are known to exhibit a high density of surface hydroxyl states.

In pending U.S. application Ser. No. 10/862,047, filed Jun. 4, 2004 (Pub. No. US 2005/0271809), and titled “Controlled Deposition of Silicon-Containing Coatings Adhered by an Oxide Layer”, and in pending continuation-in-part U.S. application Ser. No. 10/996,520, filed Nov. 23, 2004 (Pub. No. US 2005/0271893), and titled “Controlled Vapor Deposition of Multilayered Coatings Adhered by an Oxide Layer” Kobrin et al. described methods of depositing functional self-assembled-monomer (SAM) coatings using oxide films as adhesion layers on various substrates. The minimum thickness of the silicon dioxide adhesion layers grown by molecular vapor deposition (MVD) was dependent on the substrate material used. Films as thick as 200 Å were required in the case of some materials to assure their relative stability upon water immersion. PEG-comprising film attachment over oxide adhesion layers was subsequently described in pending U.S. application Ser. No. 11/123,487, filed May 5, 2005 (Pub. No. US 2006-0251795), and titled: “Controlled Vapor Deposition of Biocompatible Coatings for Medical Devices.”

In some instances, the use of a silicon oxide adhesion layer, for example, does not provide the surface properties desired subsequent to attachment of the bio-compatible coating. This is particularly true with respect to hydrolytic, mechanical, and thermal stability. It would be highly desirable to be able to form stable functionalized PEG-coated substrates where the functionalized PEG derivative is attached to a plastic or metal substrate, for example, without the use of underlying adhesion films. Both adhesion of the functional layer to the substrate material, as well as the anti-fouling functionality of the PEG layer have to meet the demanding requirements in commercial applications. Deposition of functional nano-coatings directly on materials other than those exhibiting high density of hydroxyl groups requires use of very reactive precursor head groups which are able to form bonds with the material surface. Precursors which contain silanes are not recommended for deposition from a liquid phase under ambient conditions, due to high reactivity with moisture and ambient air. Further, viscosity considerations and capillary action effects cause problems during deposition from a liquid phase, which prevent obtaining a uniform coating thickness over substrates which exhibit a complex surface geometry.

### SUMMARY

Embodiments of the present invention relate to particular precursor materials which are used to form durable hydrophilic, often biocompatible, surface coatings on plastic and metal surfaces (as well as on other surfaces which are not as difficult to adhere to). Embodiments of the invention also relate to a specialized method of vapor deposition which is used in combination with the precursor materials to deposit a coating. Finally, embodiments of the invention relate to coated articles, where the durability of the coating on the article surface is outstanding because of the manner in which the coating material is attached to a surface of the article.

There is a need for hydrophilic films and, in particular, for relatively neutral biocompatible surfaces that resist adhesion and growth of protein, lipids, and bacteria. These performance characteristics improve the performance and lifetime of articles coated with the hydrophilic films. Derivatized PEG films/coatings may employ a variety of “end-cap” groups. Examples of such derivatized PEGs, include, for example, PEG-alkyl ether, PEG-acrylate, PEG-methacrylate, PEG-amine, PEG-aldehyde, PEG-NHS-ester, PEG-maleimide, and PEG-thiol, not by way of limitation. One PEG-derivatized material which has been used to advantage is PEG alkyl ether, where the methyl ether derivative works particularly well. An alternative substituent group may be ethyl, or propyl or another similar group. In instances where a coated article is permanently or semi-permanently implanted within a living body, not only is it helpful when the coated article is resistant to bacterial growth and infection, but at least a portion of the article needs to be hydrophilic to allow for efficient binding of water and sufficient flow of oxygen to the area of the body in which the coated article may be placed. In many applications where the wear on the coating is likely to occur due to mechanical contact or where fluid flow is to occur over the substrate surface on which the layer of coating is present, it is helpful to have the coating chemically bonded directly to the substrate surface. In addition, particular precursor materials may be selected which are known to provide particular functional moieties.

Embodiments of the present invention permit the attachment of PEG-derived moieties to various plastics and metals in a manner which provides a durable hydrophilic biocompatible surface coating. (A PEG-derived moiety is a specific section of a molecule, usually complex, which provides the characteristic chemical effect or property attributed to a PEG which has been derivatized to provide a particular function.) The ability of the coating to bind with numerous plastic surfaces and to metal surfaces is due to the presence of a functional attachment group which provides an advantage over coatings previously known in the art. A plastic or metal substrate is not a limitation on the potential applications for the coatings, since the coating/film applied in accordance with the present invention binds well to other surfaces such as glass or silicon.

The coating precursors which allow functional PEG-derived coatings to be deposited on various plastic and metal surfaces are selected to present at least one amine functional attachment group which attaches to the substrate surface. The functional attachment group is typically attached to a PEG molecule at the opposite end from the PEG-derived moiety. In particular the functional groups used for attachment include tris(dimethylamino), dimethyl(dimethylamino), methylbis(dimethylamino), tris(diethylamino), dimethyl(diethylamino), methylbis(diethylamino), and combinations thereof. Amino silane functional groups work well. Use of these amino-based functional attachment groups at one position on the PEG-comprising precursor molecule, with the PEG-derived moiety at another position on the precursor molecule permits direct application of the coating over plastics and over metals directly, without the use of a catalytic agent or an intermediate adhesion layer applied over the substrate to be coated.

One embodiment PEG-comprising precursor material which performs particularly well with respect to bonding to various plastics is 2-[Methoxy(polyethyleneoxy)propyl]tris(dimethylamino)silane. This PEG-comprising precursor material illustrates the high reactivity with a plastic surface such as polystyrene, polyethylene, polypropylene, polymethyl methacrylate, polyacrylates, and acrylic-containing photoresist material, by way of example and not by way of limitation.

Biocompatible PEG coatings formed from precursor materials of the kind described above are formed by direct vapor deposition on a substrate. A vapor deposition method which is particularly advantageous is the method described in U.S. patent application Ser. No. 11/295,129 (Pub No. US 2006-0088666 A1), which was previously incorporated herein by reference. In addition, a detailed description of one apparatus embodiment which may be used to deposit the coatings appears in U.S. application Ser. No. 10/912,656 (Pub. No. US 2006-0029732 A1), assigned to the assignee of the present application. This application describes processing apparatus which can provide specifically controlled, accurate delivery of precise quantities of reactants to the process chamber, as a means of improving control over a coating deposition process. The deposition method (or process) has been referred to within the vapor deposited coating industry by the trademark MVD®. The deposition method is described in more detail in the Detailed Description of Exemplary Embodiments which follows. The apparatus used to deposit the coatings is available from Applied Microstructures, Inc. of San Jose, Calif. Depending on the processing conditions, the PEG-comprising films can be tailored to form a self-assembled monolayer or a non-self assembled film, such as an amorphous PEG-comprising film, on the substrate surface. Further, the method of application, combined with the precursor chemistries mentioned above permit a functional PEG-comprising film to be deposited at low temperatures, in the range of 20° C. to about 100° C. In typical experimental embodiments of the present invention, PEG-derived precursor materials deposited using the MVD® method have exhibited a molecular weight ranging from about 270 to about 900 (excluding the functional attachment group). The controlling factor with respect to molecular weight is whether a particular PEG-derived precursor material will have a sufficiently high vapor pressure under the process conditions at which the coating is to be deposited.

As previously discussed, one of the most important embodiments of the invention is the application of these durable, biocompatible PEG-comprising coatings over surfaces which exhibit a low density of hydroxyl groups, such as plastics and particular metals.

The combination of chemical precursors with the method of application of the coatings is particularly valuable in applications such as Bio-MEMS, Bio-arrays, medical diagnostics, medical implants, microfluidics, pharmaceutical testing devices, and other applications where conventional PEG deposition methods and chemistries do not provide an exterior surface which meets requirements pertaining to anti-fouling and biocompatibility with bodily tissue, fluids, or biological reagents.

### BRIEF DESCRIPTION OF THE DRAWINGS

FIGS. 1A-1C show PEG comprising precursor materials which include mPEG moieties and various functional attachment groups. These precursor materials were evaluated as part of the present experimentation.

FIG. 1A shows the schematic structure for 2-[methoxy(polyethyleneoxy)propyl]trichlorosilane, one of the precursor materials discussed in U.S. application Ser. No. 11/295,129 (Pub No. US 2006-0088666 A1).

FIG. 1B shows a schematic structure for 2-[methoxy(polyethyleneoxy)propyl]tris(dimethylamino)silane, one of the precursor materials which is an embodiment of the present invention.

FIG. 1C shows the schematic structure for 2-[methoxy(polyethyleneoxy)propyl]trimethoxysilane, one of the precursor materials discussed in U.S. application Ser. No. 11/295,129 (Pub No. US 2006-0088666 A1).

FIG. 2 shows a graph of the contact angle on axis 204 for the substrate which is designated fresh 220, plasma treated 230, and processed 240 after application of a vapor deposited coating over the substrate, on axis 202. The coating was deposited from the precursor material shown in FIG. 2B onto a variety of different substrates, which are shown on the graph.

FIG. 3 shows a graph of the water contact angle on axis 304 as a function of the time the coated substrate was immersed in distilled water on axis 302. The coating was deposited from the precursor material shown in FIG. 2B onto several of the substrate types shown in FIG. 2.

### DETAILED DESCRIPTION OF EXEMPLARY EMBODIMENTS

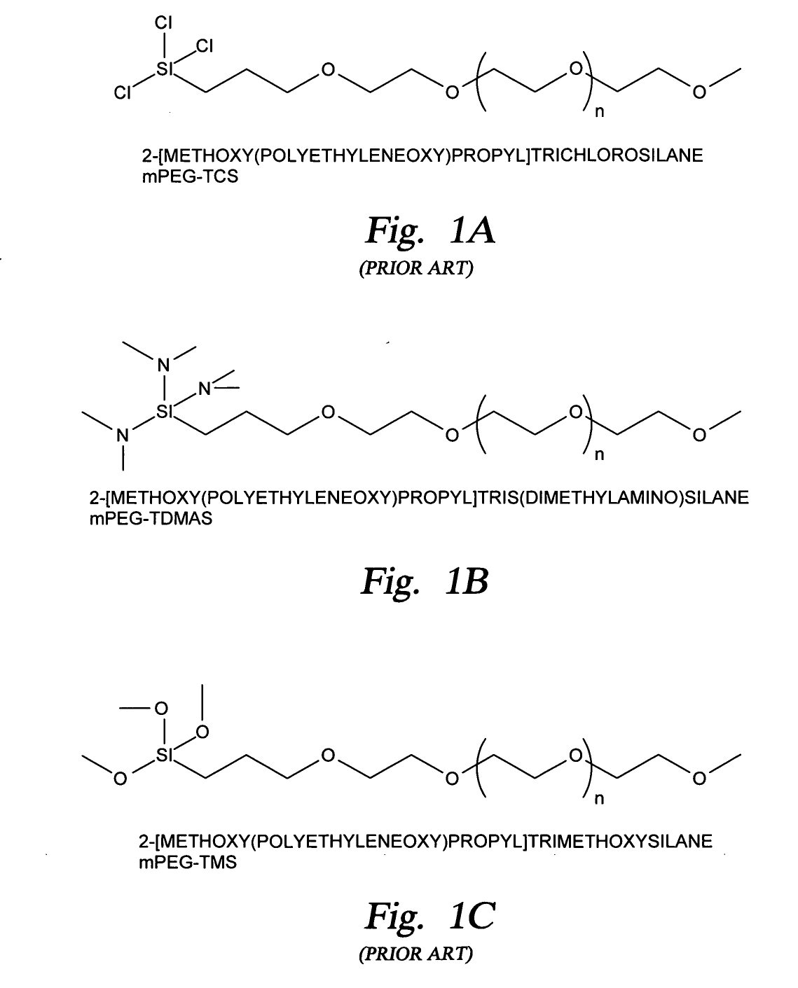

In our experimentation, we evaluated three different PEG-comprising precursor materials for the formation of coatings/films on a variety of different substrates. The portion of the precursor molecule which was functionalized to attach to the substrate included a functional group selected from the following: tricholrosilane, tris(dimethylamino)silane, or trimethoxysilane. The molecular structures for each of the PEG-comprising precursor materials is illustrated in FIGS. 1A, 1B, and 1C, respectively. Table One, below lists each of these precursor materials and the abbreviation which is used to refer to the precursor material herein.

TABLE ONE

| PEG | | |
|---|---|---|
| moiety | | |
| Type | Precursor Chemistry | Abbrev. |
| m-PEG | 2-[Methoxy(polyethyleneoxy)propyl]tris(dimethyl- | TDMAS |
| | amino)silane | |
| m-PEG | 2-[Methoxy(polyethyleneoxy)propyl]trichlorosilane | TCS |
| m-PEG | 2-[Methoxy(polyethyleneoxy)propyl]trimethoxy- | TMS |
| | silane | |

### Example One

### Comparative Example

The three mPEG silanes listed in Table One were deposited in the MVD100 system available from Applied MicroStructures, San Jose, Calif. A remotely-generated oxygen plasma step of the kind known in the art was used to pre-clean the substrates prior to film deposition. PEG-comprising hydrophilic biocompatible self-assembled monolayer coatings were deposited from the listed precursor materials by the molecular vapor deposition (MVD®) method referenced previously herein with respect to published, pending patent applications. The deposition reaction parameters were as follows.

As described in (Pub No. US 2006-0088666 A1), titled: “Controlled Vapor Deposition of Biocompatible Coatings Over Surface Treated Substrates”, which was mentioned above, an improved vapor-phase deposition method and apparatus was developed for the application of films/coatings on various substrates. The method and apparatus are useful in the fabrication of bio-functional devices, Bio-MEMS devices, and in the fabrication of microfluidic devices for biological applications. The coating formation method typically employs at least one stagnation reaction, and more typically a series of stagnation reactions. In each stagnation reaction all of the reactants which are to be consumed are charged to a vapor space over the substrate to be coated and are then permitted to react in a given process step, whether that step is one in a series of steps or is the sole step in a coating formation process. In some instances, the coating formation process may include a number of individual deposition steps where repetitive reactive processes are carried out in each individual step. The apparatus used to carry out the method provides for the addition of a precise amount of each of the reactants to be consumed in a single reaction step of the coating formation process. The apparatus may provide for precise addition of quantities of different combinations of reactants during each individual deposition step when there are a series of different individual deposition steps in the coating formation process.

In addition, to the control over the amount of reactants added to the process chamber, it is important to control the order of reactant(s) introduction, the total pressure (which is typically less than atmospheric pressure) in the process chamber, the partial vapor pressure of each vaporous component present in the process chamber, and the temperature of the substrate and chamber walls. The control over this combination of variables determines the deposition rate and properties of the deposited layers. By varying these process parameters, this controls the amount of the reactants available, the density of reaction sites, and the film growth rate, which is the result of the balance of the competitive adsorption and desorption processes on the substrate surface, as well as any gas phase reactions. In some instances, depending on the substrate material, it is also important to control the cleanliness of the substrate,

The coating deposition process is carried out in a vacuum chamber where the total pressure is lower than atmospheric pressure and the partial pressure of each vaporous component making up the reactive mixture is specifically controlled so that formation and attachment of molecules on a substrate surface are well controlled processes that can take place in a predictable manner, without starving the reaction from any of the precursors. As previously mentioned, the surface concentration and location of reactive species are controlled using total pressure in the processing chamber, the kind and number of vaporous components present in the process chamber, the partial pressure of each vaporous component in the chamber, temperature of the substrate, temperature of the process chamber walls, and the amount of time that a given set of conditions is maintained.

In some instances, where it is desired to have a particularly uniform growth of the composition across the coating surface, or a variable composition across the thickness of a multi-layered coating, more than one batch of reactants may be charged to the process chamber during formation of the coating.

An important aspect of the present invention is the surface preparation of the substrate prior to initiation of any deposition reaction on the substrate surface. The hydrophilicity of a given substrate surface may be measured using a water droplet shape analysis method, for example.

By controlling the total pressure in the vacuum processing chamber, the number and kind of vaporous components charged to the process chamber, the partial pressure of each vaporous component, and the other process conditions mentioned above, the chemical reactivity and properties of the coating can be controlled. By controlling the process parameters, features such as density of film coverage over the substrate surface; chemistry-dependent structural composition; film thickness; and film uniformity over the substrate surface are more accurately controlled. Chemistry-dependent structural composition may be generated by using of a combination of layers, where different layers may have a different chemical composition. Control over process parameters makes possible the formation of very smooth films, with RMS roughness which typically ranges from about 0.1 nm to less than about 15 nm, and even more typically from about 1 nm to about 5 nm.

PEG (and derivatized PEG such as mPEG) is available in a wide range of molecular weights. The molecular weight of the PEG or PEG derivative will determine its physical characteristics (e.g., as the molecular weight increases, viscosity and freezing point increase). PEG or PEG derivative is available with varying numbers of functional (i.e., binding) groups, such as monofunctional (one binding group), difunctional (two binding groups), and multi-functional (more than two binding groups). The molecular weight and functionality of the PEG will, in combination, determine the particular applications in which it is most useful. PEGs and derivatized PEGs, including functionalized attachment groups, which are useful in the present method, typically range from about 200 to about 2000 in molecular weight.

As previously discussed, one preferred embodiment method of vapor depositing a PEG-comprising coating is by a molecular vapor deposition process performed in a vacuum. The application method steps include:

a) subjecting a surface which is planar or a surface having any one of a variety of three-dimensional shapes to a surface cleaning process to remove contaminants. Frequently, when the contaminants are organic, an oxygen-comprising plasma is used in a processing chamber which is at a subatmospheric pressure. The pressure typically ranges from about 0.01 Torr to about 1 Torr.

b) subsequently, without exposure of the substrate to ambient conditions which contaminate or react with the substrate, exposing the substrate to a reactive precursor vapor comprising a derivative PEG moiety which provides desired coating surface properties, and a functional group which is used to attach the PEG derived moiety to the substrate. The film/coating which is formed is typically selected from the group consisting of a monolayer, a self-assembled mono-layer, and a polymerized cross-linked layer.

Optionally, additional repetition of steps may be used, including a step:

c) repeating steps a) and b) or simply repeating step b) a nominal number of times, without exposing the substrate to ambient contaminants.

Although just one layer of derivatized PEG may be applied, when it is desired to increase the thickness of the derivatized PEG layer, step b) can be repeated. Typically, the derivatized PEG-comprising precursor may be charged to the reactor chamber, reacted, and then pumped down to remove byproduct and unreacted precursor material in a series of steps to increase deposited layer thickness. A series of the charge and pump down steps in the range of about 2 to about 10 is common, with a range of about 4 to about 8 being more common. Application of a series of add-on layers to increase the total thickness of the deposited derivatized PEG-comprising layer improves the uniformity over the surface of the substrate. It is generally not necessary to plasma treat the surface of the existing derivatized PEG-comprising layer prior to charging additional reactant for deposition, since the surface of the existing derivatized PEG-comprising layer is frequently easily bonded to by the newly charged PEG-comprising precursor material.

When more than one precursor material is charged to the process chamber at one time, by changing the total pressure in the process chamber and/or limiting the partial pressure of a particular reactive vaporous component so that the component is “starved” from the reactive substrate surface, a composition of a depositing coating can be “dialed in” to meet particular requirements. When a single precursor material is charged to the process chamber, by changing the total pressure in the process chamber and/or limiting the partial pressure of the reactive vaporous component, the surface coverage of the depositing coating can be dialed in to meet particular hydrophilicity requirements.

A computer-driven process control system may be used to provide for a series of additions of reactants to the process chamber in which an individual layer or a coating is being formed. This process control system typically also controls other process variables, such as (for example and not by way of limitation), total process chamber pressure (typically less than atmospheric pressure), substrate temperature, temperature of process chamber walls, temperature of the vapor delivery manifolds, processing time for given process steps, and other process parameters if needed.

With reference to the precursor materials illustrated in Table One above, the liquid precursor was vaporized and collected in a precursor reservoir prior to injection into the reaction chamber. The amount of precursor collected was that which would provide a partial pressure in the range of 0.1-0.5 Torr (typically 0.2 Torr) in the precursor reservoir.

The processing chamber temperature was within the range of 20° C.-100° C., (typically 50° C.-80° C.). The vapor injection process was typically repeated up to about four times, depending on the substrate material and its reactivity with the precursor. The surface reaction time ranged about 5 min-90 min depending on the number of vapor injections. The multiple injections were made to assure uniform coating across the substrate surface.

PEG-comprising films were deposited directly on silicon and plastic substrates from the precursors comprising alkylamino-, alkyltrichloro-, and alkyltrimethoxy-silane functional bonding moieties. The precursor chemistries are listed in Table One. The three precursor chemistries exhibit various degrees of chemical reactivity in the gas phase in the increasing order as follows:

mPEG-TMS<mPEG-TCS<mPEG-TDMAS.

The qualitative film performance results are illustrated in Table Two, below, with respect to the water contact angle obtained and the resistance to mechanical wiping with isopropyl alcohol. A well-coated substrate with a PEG-derived functional exterior surface should have a contact angle in the range of about 50° to about 60°, and the resistance to mechanical wiping with isopropyl alcohol should be at least 10-20 wiping events.

TABLE TWO
TDMAS mPEG film deposition results.

| | Adhesion to | | |
|---|---|---|---|
| | Polystyrene (PS) | | |
| | Polymethyl | | |
| | methacrylate (PM) | | |
| | Polyethylene (PE) | Adhesion to | |
| | Acrylic-containing | Polyimide (PI) | Wiping |
| Precursor | photoresist (PW) | Polycarbonate (PC) | Durability |
| TMS | Δ | X | X |
| TCS | Δ | X | Δ |
| TDMAS | ◯ | X | ◯ |

Legend:
X—failed;
A—limited performance;
◯—good adhesion and durability.
Limited performance indicates that the contact angle ranged between about 45° C. to about 65° degrees, but that the alcohol wipe or water immersion reduced the contact angle by more than 20%.

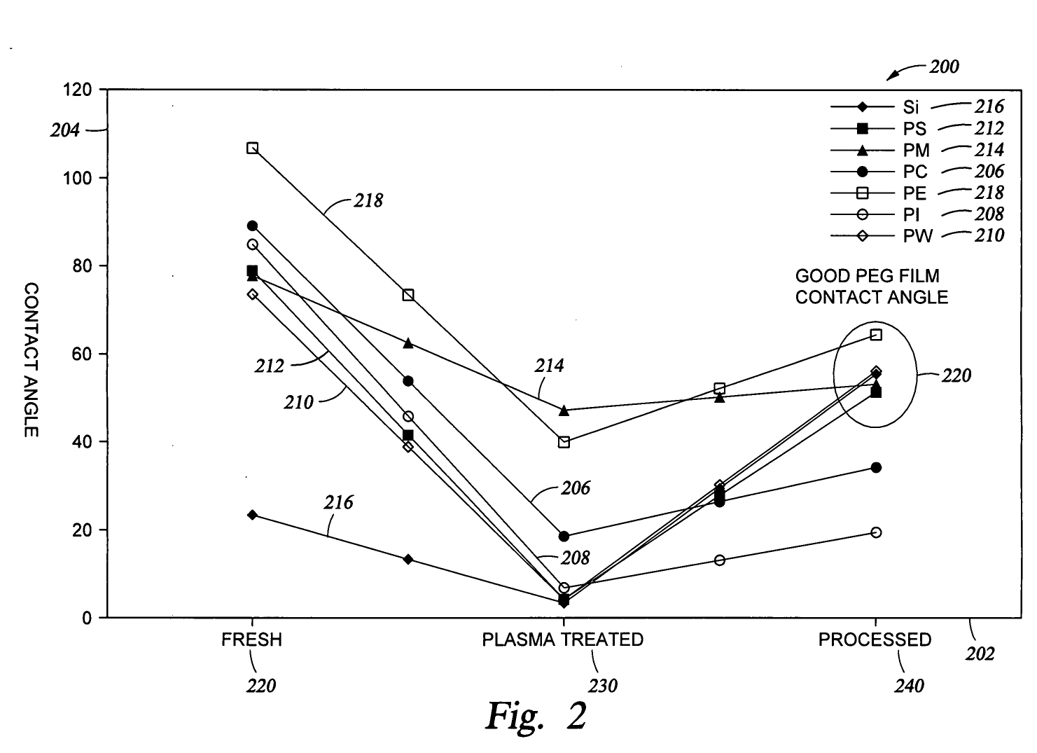

Experimentation established that TMS precursor has a lower reactivity with the substrate surface and also participates in undesirable vapor phase reactions. TDMAS was found to be highly reactive with the substrate surface, and therefore able to effectively coat materials such as polystyrene (PS), polyethylene (PE), spin-on acrylic-containing photoresist (PW), and polymethyl methacrylate (PM). The polyimide foil (PI) and polycarbonate (PC) test results indicated evidence of reduced TDMAS-PEG coupling, as indicated in Table Two and as shown in FIG. 2. However, the reduced coupling was an unexpected result and may have occurred due to substrate surface contamination prior to coating.

FIG. 2 shows a graph of the contact angle on axis 204 for the substrate indicated as “fresh” for as-received substrates 220, “plasma treated” for substrates after treatment with a remotely-generated plasma 230, and “coated” for substrates after coating 240 substrates, illustrated on axis 202. The coating was deposited from the precursor material shown in FIG. 1B onto a variety of different substrates, which are shown on the graph. Curve 206 represents a polycarbonate substrate; Curve 208 represents a polyimide foil substrate; Curve 210 represents an acrylic-comprising photoresist material; Curve 212 represents a polystyrene substrate; Curve 214 represents a polymethyl methacrylate substrate; Curve 216 represents a silicon substrate; and Curve 218 represents a polyethylene substrate. It is readily apparent that all of the processed (coated) substrates performed well in terms of providing a DI water contact angle in the desired range, from about 45° to about 65°, with the exception of the processed (coated) polyimide and polycarbonate substrates.

Although the results achievable with metal substrates are not illustrated in FIG. 2, the coated substrates are expected to provide a durable coating having the desired DI water contact angle when a deravitized PEG-comprising surface is presented. This expectation is based on adhesion tests in which other functional moieties (perfluorinates) were attached to metal using dimethylamino silanes as the functional attachment group. Metal substrates which were coated using aminosilane functional groups to adhere the coating material to the substrate included nickel, gold, aluminum, silver, platinum, and stainless steel. Uniform coatings exhibiting excellent adhesion, with a hydrophobic functional surface were created in this manner.

With respect to FIG. 2, water contact angle representation for various materials, “Fresh” indicates the “as is”/as received condition. “Plasma Treated” indicates after an RF remotely-generated oxygen plasma was used to clean the substrate in the processing chamber in which the coating was subsequently applied. “Processed” indicates the finished, coated substrate. The range of PEG films with good contact angles is circled. All PEG-comprising films were about 5 Å to about 15 Å thick.

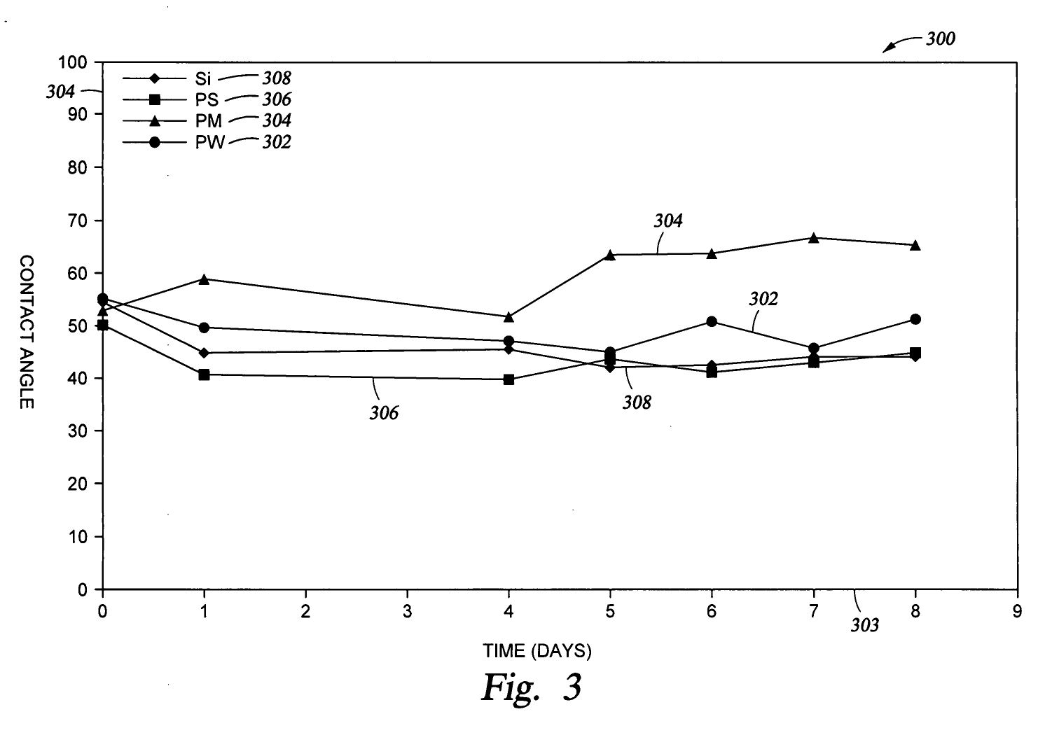

FIG. 3 shows a graph of the DI water contact angle on axis 304 as a function of the time in days the coated substrate was immersed in distilled water on axis 302. The use of dual layer films, where a PEG-comprising coating is applied over an oxide adhesion layer, allows for an improved adhesion to some substrate materials, but the resistance to immersion in water remains limited. Therefore, such films should not be relied upon for use on polymeric materials when the application requires immersion in water-based liquids of the kind frequently used in micro fluidic applications. To our surprise the use of TDMAS mPEG precursor not only improves adhesion of the functional PEG-comprising film to polymeric material substrates, but it extends surface functional durability in water over several days, as shown in FIG. 3. This figure shows the stability of the DI contact angle for mPEG-comprising films deposited on four various substrates and immersed in DI water for up to 8 days. The DI contact angle is shown on axis 304, with the time of immersion in days shown on axis 303. Curve 302 represents the immersion stability for the coating deposited from the TDMAS mPEG-precursor on a spin-on acrylic containing photoresist material substrate. Curve 304 represents the coating deposited on a poly methyl methacrylate substrate. Curve 306 illustrates the coating performance on a polystyrene substrate. Curve 308 represents the coating performance on a silicon wafer substrate. As is readily apparent, all of the coated substrates performed well, maintaining the desired DI water contact angle for the 8 day immersion period.

As previously discussed, articles coated by the derivatized PEG-comprising films using the method described herein can be used in a variety of bio-technology applications. These applications include, not by way of limitation: optical lenses, catheters, needles, implanted bone replacements, and other implanted medical devices requiring good wetting, anti-fouling properties and biocompatibility with bodily fluids. Coated substrates are also useful for diagnostics plates, bioassay devices, and components within diagnostic analytical instruments, by way of example and not by way of limitation. Other similar applications are readily apparent to one of skill in the art having read the disclosure provided herein. The PEG-comprising films may be self assembled monolayer films or may be amorphous PEG-comprising films, by way of example and not by way of limitation.

The above described exemplary embodiments are not intended to limit the scope of the present invention, as one skilled in the art can, in view of the present disclosure expand such embodiments to correspond with the subject matter of the invention claimed below.

We claim:

1. A method of providing a PEG-comprising coating on a substrate, without the use of an underlying adhesion layer, comprising:
selecting a coating precursor which comprises a PEG-derived moiety in combination with an amino silane functional group for reaction with a substrate; and
vapor depositing a coating upon said substrate from said coating precursor.

2. A method in accordance with claim 1, wherein said PEG-derived moiety is selected from the group consisting of PEG-acrylate, PEG-methacrylate, PEG-amine, PEG-aldehyde, PEG-NHS-ester, PEG-maleimide, PEG-thiol, and compatible combinations thereof.

3. A method in accordance with claim 2, wherein said PEG-derived moiety is a PEG alkyl ether.

4. A method in accordance with claim 3, wherein said PEG alkyl ether is m-PEG.

5. A method in accordance with claim 1, wherein said coating is vapor deposited onto a plasma-treated surface of said substrate, and where plasma treatment of said substrate and coating of said substrate occur without an intervening contamination of said plasma treated surface.

6. A method in accordance with claim 1 or claim 2, wherein the resultant PEG-comprising coating exhibits a DI water contact angle ranging between about 50° and about 60°.

7. A method in accordance with claim 1, wherein said coating exhibits a thickness which ranges from about 5 Å to about 50 Å.

8. A method in accordance with claim 1 or claim 2, wherein vapor depositing of said PEG-comprising coating is carried out in a subatmospheric processing chamber.

9. A method in accordance with claim 1 or claim 2, wherein said vapor depositing of said PEG-comprising coating is carried out using repeated applications of said precursor which includes said PEG-derived moiety and said amino silane-containing functional group.

10. A method in accordance with claim 1, wherein said PEG-comprising coating is a biocompatible PEG-comprising coating.

11. A method in accordance with claim 1 or claim 2, wherein said substrate comprises metal or a plastic material which is not polyimide or polycarbonate.

12. A method in accordance with claim 11, wherein said plastic material comprises a material selected from the group consisting of polyethylene, polypropylene, polyacrylate, poly(methyl methacrylate), polystyrene, acrylic-containing photoresist, silicon, and combinations thereof.

13. A method in accordance with claim 1 or claim 2, or claim 5, wherein said precursor which comprises an amino silane functional group is selected from the group consisting of tris(dimethylamino), dimethyl(dimethylamino), methylbis(dimethylamino), tris(diethylamino), dimethyl(diethylamino), and methylbis(diethylamino).

14. A method in accordance with claim 13, wherein said amino silane-containing precursor is 2-[Methoxy(polyethyleneoxy)propyl]tris(dimethylamino)silane.

15. A method in accordance with claim 1 or claim 2, wherein said PEG-comprising coating is a self-assembled or partially self-assembled monolayer comprising poly(ethylene glycol).

16. A method in accordance with claim 1, claim 2, wherein said PEG-comprising coating is an amorphous layer comprising poly(ethylene glycol).

17. An article treated on at least one surface to provide a functional PEG-comprising coating on said surface in accordance with the method claimed in claim 1 or claim 2.

18. An article treated on at least one surface to provide a functional PEG-comprising coating on said surface in accordance with the method claimed in claim 4.

19. An article treated on at least one surface to provide a functional PEG-comprising coating on said surface in accordance with the method claimed in claim 5.

20. An article treated on at least one surface to provide a functional PEG-comprising coating on said surface in accordance with the method claimed in claim 7.

21. An article treated on at least one surface to provide a functional PEG-comprising coating on said surface in accordance with the method claimed in claim 8.

22. An article treated on at least one surface to provide a functional PEG-comprising coating on said surface in accordance with the method claimed in claim 9.

23. An article treated on at least one surface to provide a functional PEG-comprising coating on said surface in accordance with the method claimed in claim 10.

24. An article treated on at least one surface to provide a functional PEG-comprising coating on said surface in accordance with the method claimed in claim 12.

25. An article treated on at least one surface to provide a functional PEG-comprising coating on said surface in accordance with the method claimed in claim 13.

26. A Bio-MEMS device comprising a hydrophilic functional surface coating applied in accordance with the method of claim 13.

27. A medical implant or device comprising a hydrophilic functional surface coating applied in accordance with the method of claim 13.
